(19) United States
(12) Patent Application Publication
(10) Pub. No.: US 2025/0213543 A1
(43) Pub. Date: Jul. 3, 2025

(54) AN EMBRYONIC ECTODERM DEVELOPMENT INHIBITOR FOR THE TREATMENT OF SICKLE CELL DISEASE

(71) Applicant: Fulcrum Therapeutics, Inc., Cambridge, MA (US)

(72) Inventor: Geoff Allen, Cambridge, MA (US)

(21) Appl. No.: 18/852,344

(22) PCT Filed: Mar. 29, 2023
(86) PCT No.: PCT/US2023/016778

§ 371 Date: Sep. 27, 2024

Related U.S. Application Data
(60) Provisional application No. 63/325,078, filed on Mar. 29, 2022.
(60) Provisional application No. 63/335,052, filed on Apr. 26, 2022.

(51) Int. Cl. A61K 31/444; A61P 7/06

(52) U.S. Cl. CPC A61K 31/444; A61P 7/06

(57) ABSTRACT

Provided herein is a method of treating a blood disorder such as sickle cell disease or a β-hemoglobinopathy with an Embryonic Ectoderm Development (EED) inhibitor as described herein.

### CROSS-REFERENCE

This application claims priority to U.S. Provisional Application No. 63/325,078 filed Mar. 29, 2022, and U.S. Provisional Application No. 63/335,052 filed Apr. 26, 2022, each of which is incorporated herein by reference in its entirety.

### BACKGROUND

Polycomb group (PcG) proteins are a family of chromatin modifying enzymes that play a key role in gene expression and are dysregulated in many human diseases. The PcG family includes two classes of Polycomb Repressive Complexes (PRCs), namely Polycomb Repressive Complex 1 (PRC1) and Polycomb Repressive Complex 2 (PRC2). PRC2 includes SUZ12 (suppressor of zeste 12), EED (embryonic ectoderm development) and the catalytic subunit, EZH2 (enhancer of zeste homolog 2), and represses genes by methylating histone H3 lysine 27 (H3K27me3) at and around the promoter regions of genes. This critical component of chromatin regulation is involved in modulation of gene transcription and plays crucial function in development, differentiation, and regeneration. Although EZH2 is the catalytic subunit, PRC2 minimally requires EED and SUZ12 for its methyltransferase activity. EED, SUZ12 and EZH2 have been found to be overexpressed in many cancers, which include but are not limited to hepatocellular carcinoma, breast cancer, prostate cancer, etc. Activating mutations in EZH2 have been found in FL (follicular lymphoma) and DLBCL (diffuse large B cell lymphoma) patients. EED normally mediates repression of gene activity by binding to di- and trimethylated lysine 27 of histone 3 where it allosterically activates EZH2 activity of PRC2. EED has also been reported to recruit PRC1 to H3K27me3 loci and to enhance PRC1 mediated H2A ubiquitin E3 ligase activity.

Taken together, EED is a critical regulator of PRC2 in the silencing of expression of genes and gene clusters involved in development including but not limited to fetal orthologues (i.e. gamma globin), Hox genes, X chromosome inactivation, etc. Thus, EED provides a pharmacologic target for the treatment of diseases or disorders to impact transcription of specific target genes in blood and other tissues.

A need exists for small molecules that modulate EED, EHZ2, and/or PRC2.

### SUMMARY

The present disclosure provides methods of treating blood disorders such as sickle cell disease or a β-hemoglobinopathy with an EED inhibitor described herein.

In an embodiment of the disclosure, provided herein is a method of treating sickle cell disease in a patient in need thereof, comprising administering to the patient, once daily, a therapeutically effective amount of a compound represented by:

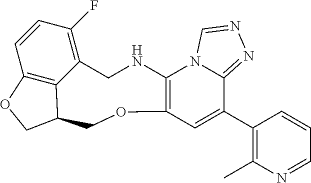 [SMILES: Cc1ncccc1-c1cc2c(n3cnnc13)NCc1c(F)ccc3c1[C@H](CO3)CO2]

or a pharmaceutically acceptable salt thereof.

In another embodiment of the disclosure, provided herein is a method of treating a β-hemoglobinopathy in a patient in need thereof, comprising administering to the patient, once daily, a therapeutically effective amount of a compound represented by:

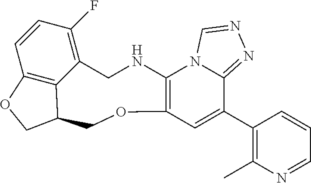 [SMILES: Cc1ncccc1-c1cc2c(n3cnnc13)NCc1c(F)ccc3c1[C@H](CO3)CO2]

or a pharmaceutically acceptable salt thereof.

### BRIEF DESCRIPTION OF THE DRAWINGS

FIG. 1 depicts plots of HBG mRNA fold induction (normalized to HBB) in patients receiving various doses of Compound 1 at safety follow up measurements.

FIG. 2 depicts plots of F-reticulocyte fold increase (% RBCs) in patients receiving various doses of Compound 1 at safety follow up measurements.

FIG. 3A depicts percentage HbF measured by HPLC for subjects receiving 6 mg of Compound 1 orally once daily. FIG. 3B depicts changes in HbF, measured by absolute percentage change from baseline, for subjects receiving 6 mg of Compound 1 orally once daily.

FIG. 3C depicts percentage HbF measured by HPLC for a subject receiving 6 mg of Compound 1 orally once daily with hydroxyurea (HU). FIG. 3D depicts pentage HbF measured by HPLC for a subject receiving 6 mg of Compound 1 orally once daily without HU.

FIG. 4A depicts percentage HbF measured by HPLC for subjects receiving 2 mg of Compound 1 orally once daily. FIG. 4B depicts changes in HbF, measured by absolute percentage change from baseline, for subjects receiving 2 mg of Compound 1 orally once daily.

FIG. 5A shows the the red cell distribution widths, or RDW, for subjects receiving 6 mg of Compound 1 orally once daily. FIG. 5B depicts total bilirubin for subjects receiving 6 mg of Compound 1 orally once daily.

FIG. 6A depicts absolute reticulocyte count in subjects receiving 6 mg of Compound 1 orally once daily. FIG. 6B depicts total hemoglobin for subjects receiving 6 mg of Compound 1 orally once daily.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

“Individual,” “patient,” or “subject” are used interchangeably herein and include any animal, including mammals, including mice, rats, other rodents, rabbits, dogs, cats, swine, cattle, sheep, horses, or primates, and humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods described herein is desirably a mammal in which treatment of a disorder described herein is desired, such as a human.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

As used herein, “treating” includes any effect, e.g., lessening, reducing, modulating, or eliminating, that results in the improvement of the condition, disease, disorder and the like.

“Therapeutically effective amount” includes the amount of the subject compound that will elicit the biological or medical response of a tissue, system, animal or human that is being sought by the researcher, veterinarian, medical doctor or other clinician. Alternatively, a therapeutically effective amount of a compound is the quantity required to achieve a desired therapeutic and/or prophylactic effect, such as an amount which results in the prevention of or a decrease in the symptoms associated with the condition.

### Compounds and Compositions

In one embodiment, an EED inhibitor of the present disclosure is Compound 1 or a pharmaceutically acceptable salt thereof. “Compound 1” refers to a compound represented by:

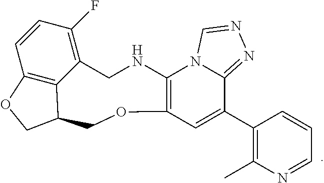 [SMILES: Cc1ncccc1-c1cc2c(n3cnnc13)NCc1c(F)ccc3c1[C@H](CO3)CO2]

A compound described herein, can be formulated as a pharmaceutical composition using a pharmaceutically acceptable carrier and administered by a variety of routes. In some embodiments, such compositions are for oral administration. In some embodiments, compositions formulated for oral administration are provided as tablets. In some embodiments, such compositions are for parenteral (by injection) administration. In some embodiments, such compositions are for transdermal administration. In some embodiments, such compositions are for topical administration. In some embodiments, such compositions are for intravenous (IV) administration. In some embodiments, such compositions are for intramuscular (IM) administration. Such pharmaceutical compositions and processes for preparing them are well known in the art. See, e.g., REMINGTON: THE SCIENCE AND PRACTICE OF PHARMACY (A. Gennaro, et al., eds., 19^{th} ed., Mack Publishing Co., 1995).

### Methods of Use

In one embodiment, described herein is a method of treating sickle cell disease in a patient in need thereof, comprising administering to the patient, once daily, a therapeutically effective amount of a compound represented by:

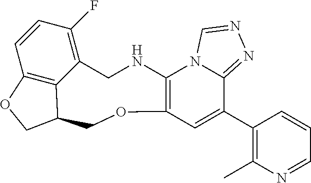 [SMILES: Cc1ncccc1-c1cc2c(n3cnnc13)NCc1c(F)ccc3c1[C@H](CO3)CO2]

or a pharmaceutically acceptable salt thereof. In some embodiments, the method comprises administering to the patient about 2 mg to about 60 mg of the compound once daily. In some embodiments, the method comprises administering to the patient about 6 mg to about 30 mg of the compound once daily. In some embodiments, the method comprises administering to the patient about 2 mg to about 30 mg of the compound once daily. In some embodiments, the method comprises administering to the patient about 6 mg of the compound once daily. In some embodiments, the method comprises administering to the patient about 20 mg of the compound once daily. In some embodiments, the method comprises administering to the patient about 2 mg of the compound once daily. In some embodiments, the method comprises administering to the patient about 12 mg of the compound once daily. In some embodiments, the method comprises administering the compound to the patient once daily for 14 days. In some embodiments, the method comprises orally administering the compound to the patient.

In another embodiment, provided herein is a method of treating a β-hemoglobinopathy in a patient in need thereof, comprising administering to the patient, once daily, a therapeutically effective amount of a compound represented by:

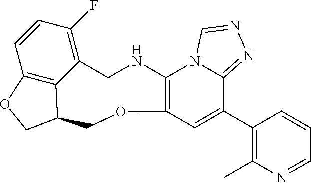 [SMILES: Cc1ncccc1-c1cc2c(n3cnnc13)NCc1c(F)ccc3c1[C@H](CO3)CO2]

or a pharmaceutically acceptable salt thereof. In some embodiments, the method comprises administering to the patient about 2 mg to about 60 mg of the compound once daily. In some embodiments, the method comprises administering to the patient about 6 mg to about 30 mg of the compound once daily. In some embodiments, the method comprises administering to the patient about 2 mg to about 30 mg of the compound once daily. In some embodiments, the method comprises administering to the patient about 6 mg of the compound once daily. In some embodiments, the method comprises administering to the patient about 20 mg of the compound once daily. In some embodiments, the method comprises administering to the patient about 2 mg of the compound once daily. In some embodiments, the method comprises administering to the patient about 12 mg of the compound once daily. In some embodiments, the method comprises administering the compound to the patient once daily for 14 days. In some embodiment, the β-hemoglobinopathy is β-thalassemia. In some embodiments, the method comprises orally administering the compound to the patient.

### EXAMPLES

### Example 1. Safety and Pharmacokinetics of Compound 1

This is a study of the safety, tolerability, pharmacokinetics (PK) and pharmacodynamics (PD) of Compound 1 from a Phase 1, single ascending dose (SAD) and multiple ascending dose (MAD) clinical study.

Healthy volunteers received oral placebo vs. single oral doses of Compound 1 in the SAD cohorts, or oral placebo vs. single oral doses of Compound 1 once daily (QD) for 14 consecutive days in the MAD cohorts. Safety assessments included AEs, vital signs, ECGs, and laboratory parameters. PK/PD blood sampling was performed pre-dose and Days 1 and 2 in the SAD cohorts, and Days 1, 2, 7, 8 and 12-15 in the MAD cohorts. Target engagement was assessed as change from baseline in H3K27me3/Total Histone H3 ratio. PD assessments included changes in F reticulocytes and HBG mRNA.

Compound 1 was evaluated in 7 healthy volunteer (HV) SAD cohorts (2 to 60 mg), 5 HV MAD cohorts (2 to 30 mg QD, 14-day dosing). SAD dose cohorts include 2 mg, 4 mg, 6 mg, 10 mg, 20 mg, 30 mg, 40 mg, and 60 mg Compound 1. MAD dose cohorts include 2 mg, 4 mg, 6 mg, 10 mg, 20 mg, and 30 mg Compound 1. Treatment-related adverse events were mild (Grade 1 or 2). There have been no SAEs or discontinuations. PK profiles demonstrated dose proportionality across all doses. Robust target engagement was observed across the entire dose range. Additionally, exposures achieved in the 6-30 mg MAD cohorts resulted in 2.4-6.2 fold placebo-adjusted increases from baseline in HBG mRNA.

Compound 1 was generally well tolerated and no unexpected safety signals were detected.

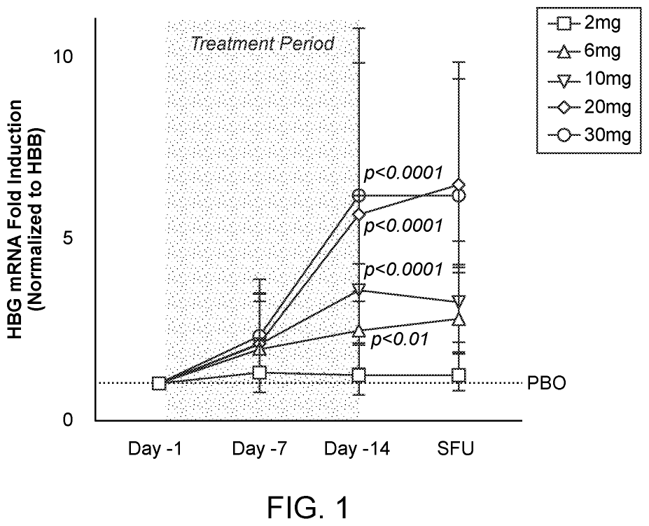

FIG. 1 shows that HBG mRNA induction is both time- and dose-dependent in Compound 1 MAD cohorts. Safety Follow-up (SFU) samples were collected 7-10 days post 14-day treatment period.

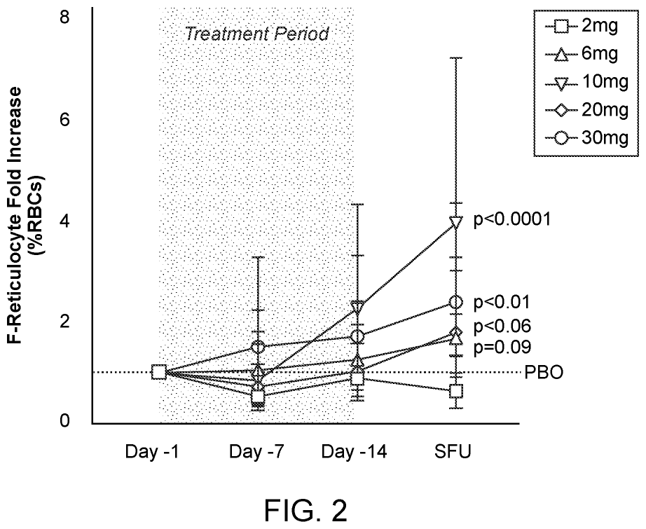

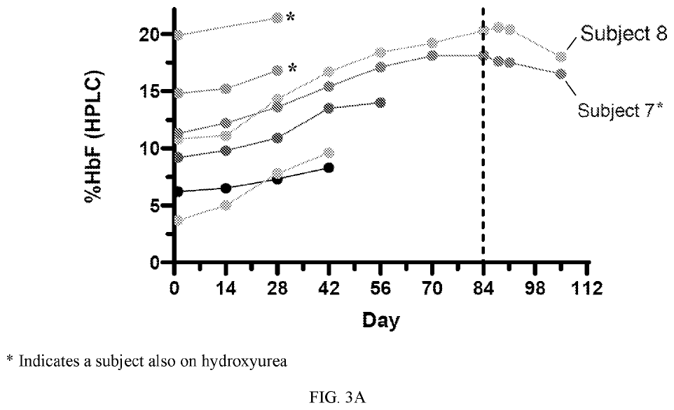

FIG. 2 shows that F-reticulocytes do not predict HbF fold induction, but demonstrate translation of HBG to HbF. A mean 1.7-3.9 fold increase in F-reticulocytes was observed in safety follow-up (SFU) visits from patients dosed at ≥6 mg Compound 1. Increases in F-cells and HbF protein are projected with longer treatment duration.

Data in FIG. 1 and FIG. 2 are presented as geometric LS mean ratios (to placebo) with a 95% confidence interval. Mixed-effects model for repeated measures (MMRM) analysis was performed for Day 7 and Day 14 measurements. An analysis of covariance (ANCOVA) was utilized for SFU data. In FIG. 1 and FIG. 2, “PBO” is placebo.

### Example 2. A Phase 1 Open-Label, Multiple-Dose Study to Evaluate Safety and Tolerability, Pharmacokinetics and Pharmacodynamics of Compound 1 in Subjects with Sickle Cell Disease (SCD)

This is a Phase 1 multicenter, open-label study evaluating the safety, tolerability, pharmacokinetics (PK), fetal hemoglobin (HbF) induction and biological activity of Compound 1 in subjects 18-65 years of age with sickle cell disease (SCD). This study comprises 2 parts and is being conducted in subjects 18-65 years of age with sickle cell disease (SCD) either taking hydroxyurea (HU) at a stable dose for at least the preceding 3 months, or not taking hydroxyurea (HU).

The study consists of two phases. In Part A, Cohort 1 received 6 mg of Compound 1 by mouth once daily for 4 weeks, Cohort 2 received 2 mg of Compound 1 by mouth once daily for 4 weeks, and Cohort 3 received 12 mg of Compound 1 by mouth once daily for 4 weeks. An additional cohort may receive up to and including 20 mg of Compound 1 by mouth once daily for 4 weeks depending on the Data Monitoring Committee [DMC] review. Additional cohorts (up to five total cohorts) may be added based on the safety and pharmacokinetic data observed in subjects from the prior and ongoing cohorts. Up to 10 subjects have been or will be enrolled in each treatment arm.

Part B is the extension study. Eligible subjects who consent to continue in the extension study continue on treatment at the same dose level selected in Part A for an additional 8 weeks. Subjects who decline to participate in the extension study are followed for an additional 2-3 weeks.

The primary endpoints of the study are to evaluate the safety and tolerability of Compound 1 as measured by the frequency of adverse events and to evaluate single and multiple-dose pharmacokinetics of Compound 1 in subjects with sickle cell disease. Secondary endpoints include evaluating the effect of Compound 1 on fetal hemoglobin induction in peripheral blood, as well as changes from baseline in reticulocytes, in subjects with sickle cell disease. Exploratory endpoints include biomarkers of hemolysis, target engagement, incidence of vaso-occlusive crises (VOCs), and quality of life (QOL) measurements.

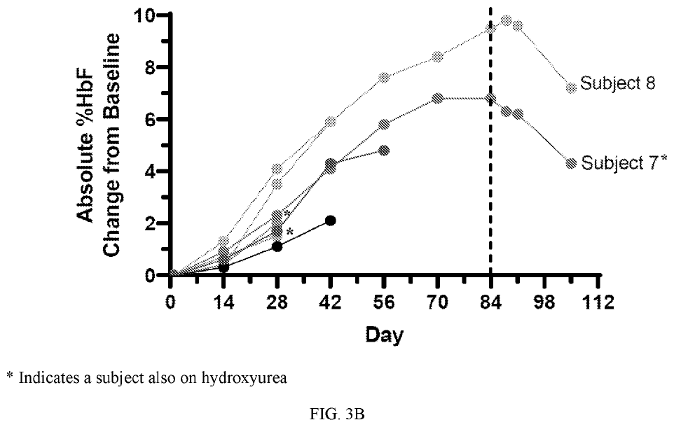

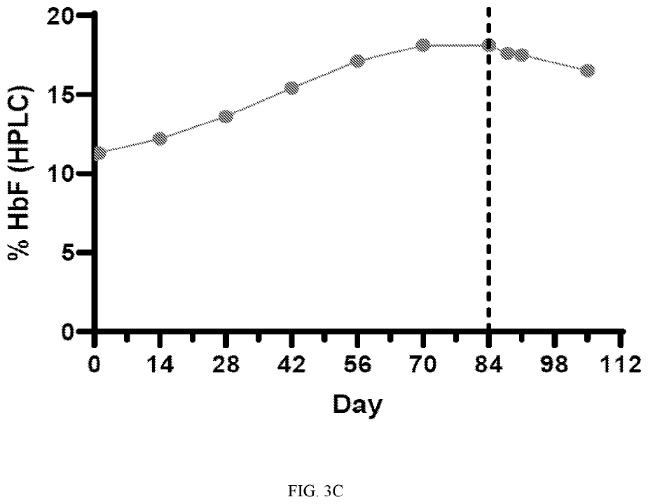

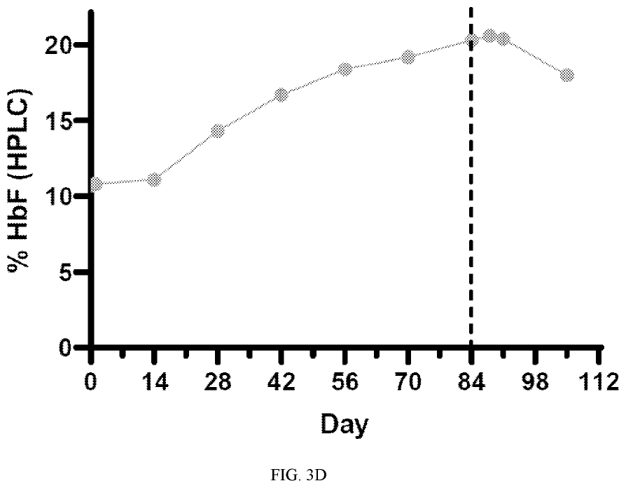

Results from the study were obtained. Overall, Compound 1 was generally well tolerated. Percentage HbF measured by HPLC for Cohort 1 subjects is provided as FIG. 3A, including for two subjects undergoing more than 84 days of treatment (Subjects 7 and 8). FIG. 3B depicts HbF absolute percentage change from baseline for subjects in Cohort 1, including for Subjects 7 and 8.

FIG. 3C depicts percentage HbF by HPLC for Subject 7 of Cohort 1 only. FIG. 3D depicts percentage HbF by HPLC for Subject 8 of Cohort 1 only. These data show that HbF increase was robust (6.8%-9.5%) at day 84 and that there were no apparent response differences in HU versus non-HU treated subjects.

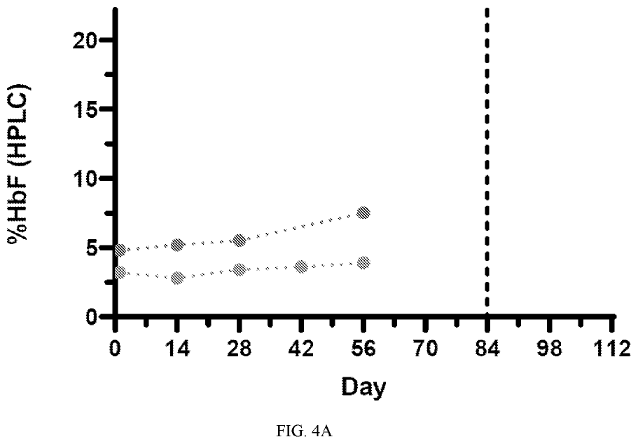

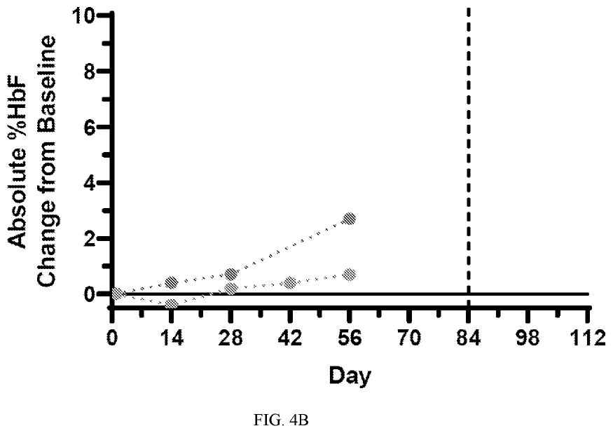

FIG. 4A depicts percentage HbF measured by HPLC for Cohort 2 subjects. FIG. 4B depicts HbF absolute percentage change from baseline for subjects in Cohort 2. These data show that Compound 1 has a dose-dependent, clinically relevant and consistent increase in HbF. Further, data from a subject in the 12 mg dose cohort (Cohort 3, n=3) showed up to 10.0% absolute HbF increases from baseline after 42 days of treatment.

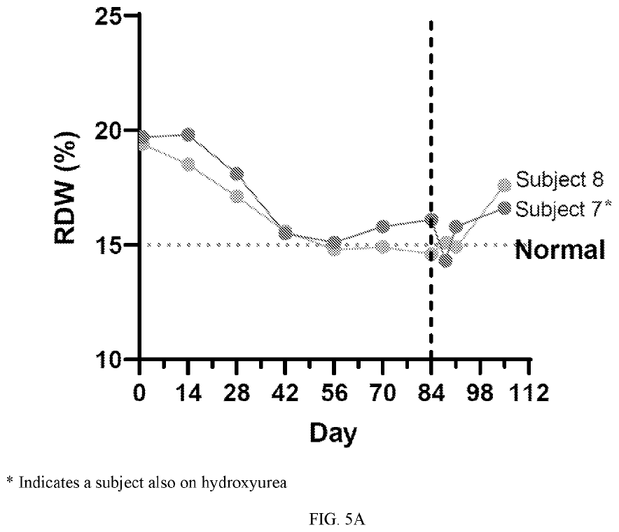

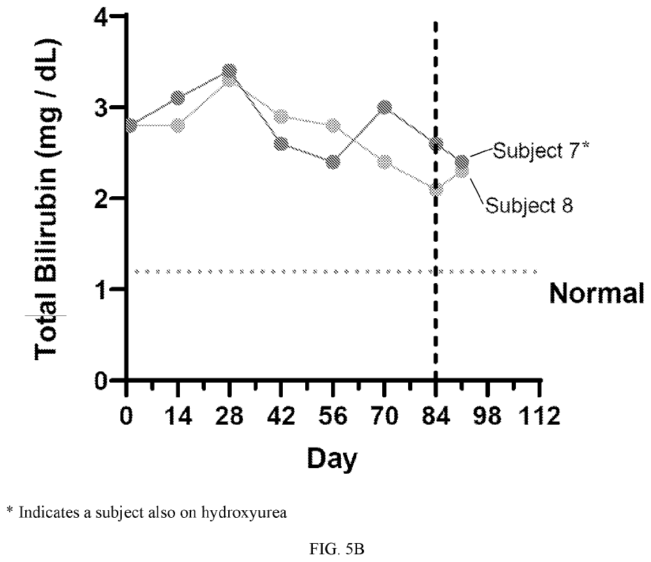

Hemolysis impact in Cohort 1 subjects: FIG. 5A shows the red cell distribution widths, or RDW, for certain Cohort 1 subjects (Subjects 7 and 8) during treatment. The data show that reductions in RDW indicate red blood cells became more uniform in shape. FIG. 5B depicts total bilirubin for Subjects 7 and 8 of Cohort 1 across treatment.

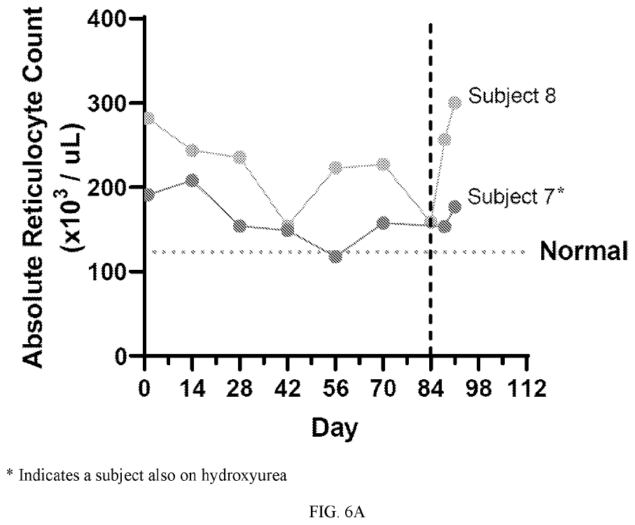

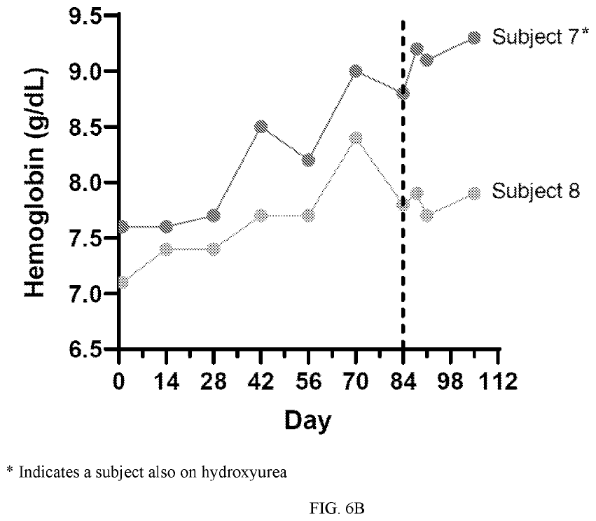

Amelioration of anemia in Cohort 1 subjects: FIG. 6A depicts absolute reticulocyte count in certain Cohort 1 subjects during treatment. FIG. 6B depicts total hemoglobin for Cohort 1 subjects across treatment. The data show that reductions in reticulocytes and increases in total hemoglobin indicate less anemia and healthier bone marrow function.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

What is claimed is:

1. A method of treating sickle cell disease in a patient in need thereof, comprising administering to the patient, once daily, a therapeutically effective amount of a compound represented by: 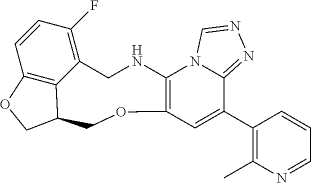 [SMILES: Cc1ncccc1-c1cc2c(n3cnnc13)NCc1c(F)ccc3c1[C@H](CO3)CO2]
or a pharmaceutically acceptable salt thereof.

2. The method of claim 1, comprising administering to the patient about 2 mg to about 60 mg of the compound once daily.

3. The method of claim 1 or 2, comprising administering to the patient about 6 mg to about 30 mg of the compound once daily.

4. The method of any one of claims 1-3, comprising administering to the patient about 2 mg to about 30 mg of the compound once daily.

5. The method of any one of claims 1-4, comprising administering to the patient about 6 mg of the compound once daily.

6. The method of any one of claims 1-4, comprising administering to the patient about 20 mg of the compound once daily.

7. The method of any one of claims 1-4, comprising administering to the patient about 2 mg of the compound once daily.

8. The method of any one of claims 1-4, comprising administering to the patient about 12 mg of the compound once daily.

9. The method of any one of claims 1-8, comprising administering the compound to the patient once daily for 14 days.

10. The method of any one of claims 1-8, comprising administering the compound to the patient once daily for 4 weeks.

11. The method of any one of claims 1-10, comprising orally administering the compound to the patient.

12. A method of treating a β-hemoglobinopathy in a patient in need thereof, comprising administering to the patient, once daily, a therapeutically effective amount of a compound represented by: 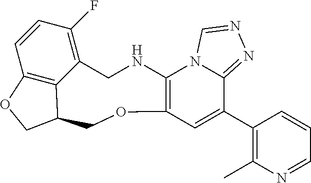 [SMILES: Cc1ncccc1-c1cc2c(n3cnnc13)NCc1c(F)ccc3c1[C@H](CO3)CO2]
or a pharmaceutically acceptable salt thereof.

13. The method of claim 12, comprising administering to the patient about 2 mg to about 60 mg of the compound once daily.

14. The method of claim 12 or 13, comprising administering to the patient about 6 mg to about 30 mg of the compound once daily.

15. The method of any one of claims 12-14, comprising administering to the patient about 2 mg to about 30 mg of the compound once daily.

16. The method of any one of claims 12-15, comprising administering to the patient about 6 mg of the compound once daily.

17. The method of any one of claims 12-15, comprising administering to the patient about 20 mg of the compound once daily.

18. The method of any one of claims 12-15, comprising administering to the patient about 2 mg of the compound once daily.

19. The method of any one of claims 12-15, comprising administering to the patient about 12 mg of the compound once daily.

20. The method of any one of claims 12-19, comprising administering the compound to the patient once daily for 14 days.

21. The method of any one of claims 12-19, comprising administering the compound to the patient once daily for 4 weeks.

22. The method of any one of claims 12-21, wherein the β-hemoglobinopathy is β-thalassemia.

23. The method of any one of claims 12-22, comprising orally administering the compound to the patient.
